(19) United States
(12) Patent Application Publication
(10) Pub. No.: US 2017/0189738 A1
(43) Pub. Date: Jul. 6, 2017

(54) Gyrokinetic Engine

(71) Applicant: Christopher Oremus, Las Vegas, NV (US)

(72) Inventor: Christopher Oremus, Las Vegas, NV (US)

(21) Appl. No.: 14/987,758

(22) Filed: Jan. 5, 2016

(51) Int. Cl. A63B 21/22; A61B 5/00; A61B 5/0205; A61B 5/11; A63B 21/00; A63B 21/005

(52) U.S. Cl. CPC A63B 21/222; A63B 21/00192; A63B 21/005; A61B 5/0205; A61B 5/11; A61B 5/742; A61B 5/0004

(57) ABSTRACT

The gyroscopic exercise device, comprised of one or more rotating mass equipped with accompanying gears, bearings, and optional components, encased in a frame connected to an attachment which contains additional optional equipment including electric motors, magnets, batteries, wiring and electronics, to which one or more handles or grips are connected in a temporary or fixed position, intended to overcome the high degree of difficulty and failure new users experience when using prior art friction-based gyroscopic exercise devices, by providing a novel driving system and rotor that is substantially more efficiency, powerful and durable with means of servicing wear points, enables users to consistently progress, further encouraging regular use, thereby developing greater strength circulation, and coordination in the hands, arms and upper body, which enhances stability and control when users operate handheld equipment.

### BACKGROUND

1. Field of Invention

This invention relates to gyroscopic exercise devices powered by kinetic energy producing resistance to movement, specifically to those that employ a rotating and precessing mass as the movement to power it.

Gyroscopic exercise devices that create resistance to movement by rotation and precession of mass operate by the same laws of physics describing conservation of angular momentum in a gyroscope, however these device are not gyroscopes in that they to not “scope” anything at all. The conservation of angular momentum energy in this example causes the axis of rotation to remain fixed in place wherever it is at like a scared child in the middle of a gun fight while it has bullets firing all around it. Angular momentum is like the momentum of a bullet but constrained to rotate in circles like a dog chasing its tail. The momentum accumulated in the speeding the mass around the perimeter of the rotating mass builds a power much stronger than its gravitational weight mass*velocity=momentum. This momentum traveling in circles behaves differently than the momentum of a bullet that will travel in a mostly straight line until deflected when it will ricochet at an angle and continue in a mostly straight line until deflected again. When the angular momentum of a rotating mass is pushed off of its axis of rotation it exhibits an unexpected behavior where it will rotate around the axis perpendicular to its rotation axis while either of those axis's are facing anywhere since this behavior does not have any relationship to gravity. The amount angular momentum increases proportionate to increases of speed or mass and with more angular momentum the force needed to deflect also increases. So a two pound rotor spinning at then thousand revolutions a minute might take fifteen times its weight in force to move it. So as a resistance exercise gyroscopic resistance is remarkably well suited as the force to work against since it works in any position or grip, anywhere on this planet or in space and even under water. Their self-contained totally portable devices weighing just a few pounds and able to fit in a small bag to travel anywhere providing cardiorespiratory and/or strength training anytime and anywhere.

The field of gyroscopic exercise is relatively young and yet to mature to its full potential once there is a device available with enough engineering invested that it will meet and exceed expectations. The present invention has been engineered to meet and exceed the demands of rigorous use and provided the ability to tune itself to its use with new technology converting the power source of kinetic energy to useful resistance to the motion that powers it.

2. Description of Prior Art

There have been known previous gyroscopic exercisers developed in the hope of providing dynamic physical exercises capable of developing beneficial physiological improvements to the user. Unfortunately after the first invention the imagination of engineers has not looked beyond the basic structure and function of the first gyroscopic exerciser invented over forty years ago. All predecessors to this design share the same basic functionality which is also the greatest weakness of all these designs and what separates them from the present invention. The prior art gyroscopic exercise devices engage the rotation of precession through friction at the tip of the axle as its dragged along a ring surrounding the rotor causing the rotor to rotate faster with each rotation of precession. The ring the axle rides upon must be perfectly smooth and provide enough friction to provide adequate traction to rotate the axle as it precesses but too much friction prevents precession and shortens its lifespan. If friction was eliminated in these prior art devices they would cease to function without a means to convert precession to rotation and as such are bound by this inherent flaw to a short lifespan. Each device model that holds a standing in the arena of gyroscopic exercise devices has its own unique weaknesses and faults however what they all have in common is their use of friction rings and the weaknesses inherent to it.

The rings that the axle slides on during precession quickly wear with necessary friction creating wear which quickly opens the space constraining the movement of the axle making activation difficult as the rotor is allowed to move in undesired ways. The debris from friction tearing material off the friction rings accumulates and creates excessive drag slowing precession rotation and rotation of the rotor and as the rings of these devices that must be perfectly smooth even the slightest imperfection will affect performance by creating knocks with each rotation of precession decreasing the efficiency of the movement. The present invention is mounted with bearings throughout eliminating and providing a sturdy base of support that is able to withstand trauma and vigorous use while providing a long and maintenance free lifespan making it vastly different than prior art.

A variety of methods have been employed to start prior art devices including a manually wound pull cord, retracting pull cord, electric motors, and other manual techniques. All of these methods become a nuisance to the user when the pull cord is lost, or when the pull starter breaks or when people just can't figure out how to get it going. In contrast to this frustration the present invention does not have or need any trinkets, tricks or accessories to get it started as the industrial strength bearing supported clutch system is engages rotations at any time the driving rotation speed is greater than the stored angular velocity of the rotor and thereby will reliably rotate the rotor from zero rotations and disengage when it is going faster than its driving force allowing the user to manually rotate the handle to start until there is adequate angular momentum to create precession where normal exercise activity would take place and thus making it vastly different than prior art which is very difficult to get going fast enough to start and doesn't perform consistently from one hour to the next.

Perhaps the most fundamental flaw of the majority prior art gyroscopic exercisers is their difficulty of use. These friction drive devices must spin at an exceptionally high rate to function as intended and it's very difficult to achieve high speed rotation by a new user that doesn't yet know how to use it or drive it to spin faster, so their stuck with a device that they can't learn how to use. When the device is started by an experienced user or external starter before being passed to the new user to try out their experience is short lived as the friction inherent to the design quickly drags the rotor speed below its stall point where it will no longer precess. Then if a user does learn to learn to use it the behavior of the device will change with each hour of use as friction and wear the device like burning the wick of a candle. To achieve even a reasonable lifespan these devices require attentive care to prevent any form of impact, any contamination of dirt or debris, they require constant cleaning even without contamination, and maintenance to replace broken parts. These factors have combined to prevent the adoption of gyroscopic exercise as a regular form of resistance exercise and relegated it to coffee table conversation topic in sharp contrast to the present invention which is designed to be withstand vigorous use consistently providing predictable performance without regular maintenance.

Further evidence of the shortcoming in the design prior art friction-based gyroscopic exercisers is their inherent fragility. Even correct, light use by an experienced person will result in fractures at stress points where load has not been correctly compensated for causing progressive wobble in the rotor until it breaks. There is also the accumulating number of clicks and clunks from dents in the friction rings that repeat with each revolution of precession until something finally breaks and the clunking finally stops. However the present invention possesses none of these potential weaknesses which have been eliminated through strategic engineering of braced structures able to resist stress with sturdy and efficient, replaceable bearings with structure designed to absorb vibration and shock in a strategically designed system ready to usher in a new era of exercise technology.

Another significant difference between the present invention and prior art, is the fact that the internal rotor space in prior art is a spherical space where rotor is able to move mostly unrestrained, whereas in the present invention it need not be. The friction driven gyroscopic devices rotate through full 360 degree rotations dragging axle around with it in a dome large enough to accommodate it. The present invention with its fully constrained and bearing supported movements does not occupy the same space moving in unison with the frame while engaging the rotary transmission as it turns, providing the same essential function but in a substantially different form enabling device to travel easily or reside within smaller embodiments such as paddles, racquets, and adapt to a wider range of uses including clubs, bats, pistols and rifles.

U.S. Pat. No. 3,726,146 by Archie L. Mishler awarded in Apr. 10, 1973, titled ‘Gyroscopic Device’ is the first example of a precession driven exercise gyroscopic wrist exerciser. While this design is the first to release the gyroscope rotors axle to spin freely through a circular race surrounding the rotor, it employs the friction of said rotor on a race as a means to convert precession to rotation of the rotor. However that friction on the race is what would cause the movement of the rotor to be hindered and inconsistent, somewhat putting in to question the functional capability of the device. These weaknesses have been addressed and overcome in the present invention, which does not use this prior art's races and friction engagement so is thus fundamentally different.

U.S. Pat. No. 4,150,580 by Jerrold W. Silkebakken awarded in Apr. 24, 1979, titled ‘Gyroscopic Exerciser’ improved on the stability and spin of the rotor as it is precessing by improving upon the rotor design reducing wear and improving function. Regardless, the friction on the race is still significant enough to shorten the lifespan and limit the performance of the device and make its use difficult for inexperienced users. This is the same basic design and operation that has persisted throughout all future embodiments and permutations of the Mishler design without any fundamental changes to the operation of the device. This sectioned ring friction drive mechanism is not employed in the present invention which uses rotational drive transmission and so is thus fundamentally different.

U.S. Pat. No. 4,703,928 by James C. Escher, awarded in Nov. 3, 1987, titled Precessional exercising device′ is a device is powered entirely by an electric motor and does not perform any conversion of precession or physical movement into rotational energy. It is supposedly intended for resistance training of the foot and hands. This device does not perform any regenerative conversion of precessional energy and is thus substantially different from the present invention.

Design U.S. Pat. No. D350,796 S by Kenneth L. Pravitz awarded in Sep. 20, 1994, titled ‘Gyroscopic Exerciser’ is a two handled gyroscopic exercise tool whose core friction drive function is the same as U.S. Pat. Nos. D351436, D351437, D365612 and D381719 by the same author each with different configurations of handles. The present invention does not employ this friction drive mechanism in any from and is functionally and fundamentally different in its use of gears and bearings.

Also by Mr. Pravitz is Design U.S. Pat. Nos. D351436 S and D351437 S both awarded in Oct. 11, 1994 discloses a plurality of friction driven exercise gyroscopes positioned at either end of the centrally located handle. These devices feature the friction driven activation in two gyroscopic exercisers simultaneously and is again fundamentally different from the present invention by method of use and power generation.

Design U.S. Pat. No. D365612 S by Kenneth L. Pravitz awarded in Dec. 26, 1995, and U.S. Pat. No. D381719 S both titled ‘Gyroscopic Exerciser’ disclose friction driven gyroscopes but this time without a handle or as an ornament, but are nearly identical to the Mishler and Silkebakken patents. The present invention does not disclose a friction driven gyroscope and is thus fundamentally different from these patents.

U.S. Pat. No. 5,800,311 A by P. S. Chuang awarded in Sep. 1, 1998, titled ‘Powerball Wrist Exerciser’ is another friction driven gyroscope exerciser to be gripped in the hand. In contrast, the present invention using gears and bearings or magnetic drive assistance to provide motion to the rotor, and is thus fundamentally different from this particular invention.

Several other patents on friction driven gyroscope exercisers invented by Yun Yu Chuang and Ming Hung Lin fundamentally different from the present invention are: U.S. Pat. No. 6,623,405 B2 awarded in Sep. 23, 2003, U.S. Pat. No. 7,077,786 B2 awarded in Jul. 18, 2006, and U.S. Pat. No. 7,381,155 B2 awarded in Jun. 3, 2008.

Design U.S. Pat. No. D418562 S by Jesus P. Ibarra and Kenneth L. Pravitz, awarded in Jan. 4, 2000 titled ‘Attachment Unit for a gyroscopic exerciser device’ discloses a foot attachment for operation of a friction driven gyroscopic exercise device. The present invention does not use friction as means to spin its rotor and does not attach to the foot for operation, and so is fundamentally different.

U.S. Pat. Nos. 6,053,846 and 6,186,914 B1 by Chien-Der Lin, awarded in Apr. 25, 2000 and Feb. 13, 2001 titled ‘Wrist Exerciser’ and ‘Wrist Ball’ respectively, both pertain to a friction driven gyroscope that uses a string or zip cord method of starting movement, and friction as means of regenerative power to continue movement none of which are employed in the present invention which uses a system of gears and clutches and is thus fundamentally different.

U.S. Pat. No. 6,234,045 B1 by Kenneth W. Kaiser, awarded in May 22, 2001, titled ‘active tremor control’ is a stabilizing device to aid accuracy in hitting a target. It is not intended in any way to provide a physiological benefit to the user. The function is actually counter to the action and intent of the kinetic precession rotor of the present invention, which is specifically designed to provide a physiological benefit to the user with regular use, and is thus clearly fundamentally different.

U.S. Pat. No. 6,770,012 B2 by Hsiu-Min Kuo awarded in Aug. 3, 2004 titled ‘Self Generating Wrist ball’ is another friction-based gyroscope exerciser but with bumps on the surface and light emitting diodes. This friction-based gyroscopic exerciser is fundamentally different from the gear and bearing or magnetic drive assisted kinetic precession rotor of the present invention.

U.S. Pat. No. 7,102,258 B2 by Kun-Tsai Shen awarded in Sep. 5, 2006 titled ‘Manual electric generating device’ is another friction-based gyroscope that has coils and magnets to generate enough electricity to power light emitting diodes embedded into the friction driven gyroscopic device. The light of the diodes will cease when the user motion is stopped, as no battery was provided in the device. Again this is fundamentally different from the present invention, which is not friction-based and uses gears and bearings as method for producing regenerative power.

U.S. Pat. No. 7,326,156 by William S. Dworzan awarded in Feb. 5, 2008 titled ‘Handheld Gyroscopic Exercise Device’ and U.S. Pat. Nos. 7,563,210 and 7,935,035 by Tom Smith awarded in Jul. 21, 2009 titled ‘Gyroscopic Total exerciser’ and “Dynamax with electric starter” awarded on May 3, 2011 respectively, are almost identical. All describe friction-based gyroscopic exercise devices having an embedded electric motor or pull start in the rotor housing which will not turn without electric current applied or pull started to initiate adequate rotation to generate precession. This device once again uses friction as its method of converting precession to rotation which is fundamentally different from the present invention, which will consistently start and continue to spin without friction or electricity through normal user interaction.

The following patents all disclose friction driven gyroscopic devices, which are fundamentally different from the gear and bearing driven or magnetic drive assisted kinetic precession rotor of the present invention:

U.S. Pat. No. 7,736,275 by Yun Yu Chuang and Ming Hung Lin awarded in Jun. 15, 2010 titled ‘Wrist exerciser with sound generator’; U.S. Pat. No. 5,150,625 by Timothy Kelliher awarded in Jun. 16, 2011 titled ‘Gyroscopic exercise ball’; U.S. Pat. No. 8,449,436 B2 by Pei Sung Chuang and Yun Yu Chuang awarded in May 28, 2013 titled ‘Wrist Exerciser with handle’.

### OBJECTS AND ADVANTAGES

Several objects and advantages of the present invention are:
a) To provide a high performance Gyrokinetic Engine with highly efficient and robust construction, made with replaceable and serviceable components, providing a broad customizable power range working reliably with relative ease compared to prior art, whereby a broader range of users are able to perform the exercises regularly thereby achieving the true value of its use and best value without machine failure powered by an efficient rotary transmission system with selective engagement of drive components to preserve energy and improve performance by reducing drag. This is in sharp contrast to prior art which quickly wears out or breaks before inexperienced have a chance to be quickly frustrated by their difficulty of use and steep learning curve inherent to all friction-based gyroscopic devices.
b) To expand the efficacy of momentum resistance training through Gyrokinetic Engines by creating devices that perform consistently and provide a long lifespan delivering a power to weight ratio and force potential greater than any predecessor while simultaneously extending its range of possibilities to provide lower power range of functionality beyond the capability of prior art by providing constantly variable speed-to-power drivetrain ratio transmitting precession rotation torque to rotating mass thereby accommodating any size user with any level of strength or experience while moving the device in either direction of precession with a transmission differential keeping the rotor spinning in the same direction as its momentum carries it. Whereas prior art devices would break if spun too fast or cease to precess if spinning too slow the present invention efficiently stores momentum in the rotating mass providing abilities never before experienced with prior art. Add an optional grip and unique angular forces can be created and a greater variety of physical challenges can be experienced, resulting in enhanced adaptive responses to the benefit both experienced and inexperienced users alike
c) To provide a Gyrokinetic Engine capable of maintaining angular momentum energy by application of stored electrical energy through electromagnetic coils maintaining speed of rotor above minimum operating speed necessary to achieve precession, to maintain speed at predetermined spin rate, or to spin the rotating mass faster than the user would be capable of doing alone. Those same coils would capture energy as electricity at times when the speed of manual precession rotation drives the rotor faster than set speed.

### SUMMARY

The present invention solves a long existing and yet unresolved need for a durable, high performance, highly efficient Gyrokinetic Engine that's easy to use providing a viable resistance training program. All of the existing prior art fragile, and expensive while providing an exceedingly short lifespan that has relegated these devices novelties and trinkets to make interesting conversation about physics but never as a serious exercise. The gyroscopic exercise activities excite new users as their amazed by its seemingly magical ability to resist movement they enthusiastically will use these devices for the short time they perform but sadly will performance will fade long before interest. The experience of consistent frustration and disappointment with prior art devices that eliminated them from consideration as a viable resistance training regime has led to the innovation that is the present invention designed to provide capabilities that prior art could not in a reliable finely tuned precision machine able to withstand the rigors of performance athletes in professional training environments. Not only is this device able to withstand the rigors of performance athletes its light and versatile enough to seamlessly integrate into your lifestyle or seamlessly transition from a challenging workout in the hand of an Olympic athlete into the hand of a child or elderly person for a gentle workout with a device adapt to dynamically adapt to each person's individual strength level and exercise speed.

### DRAWINGS

### Drawing Figures

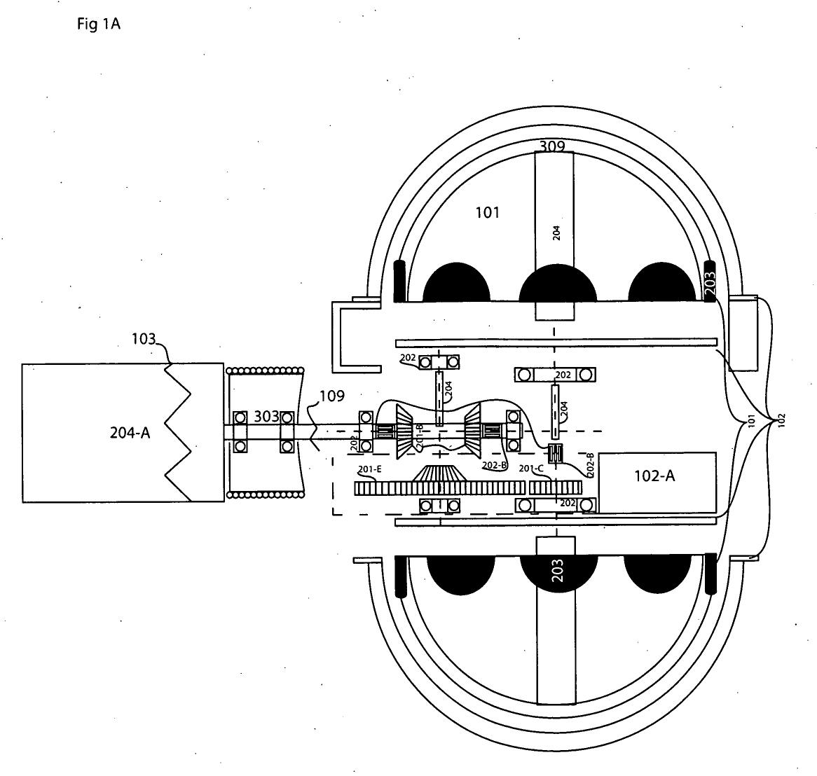

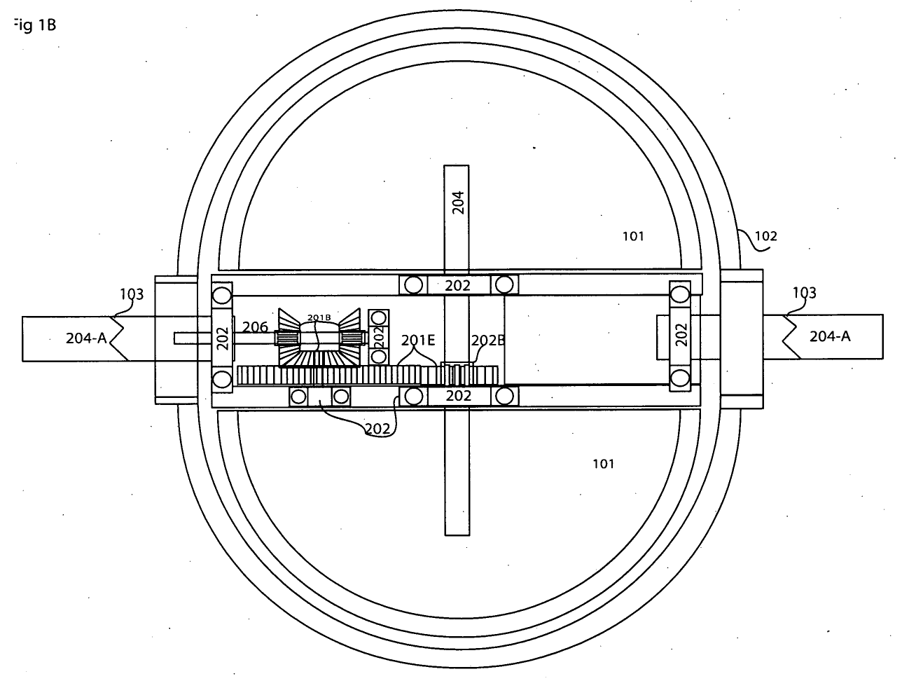

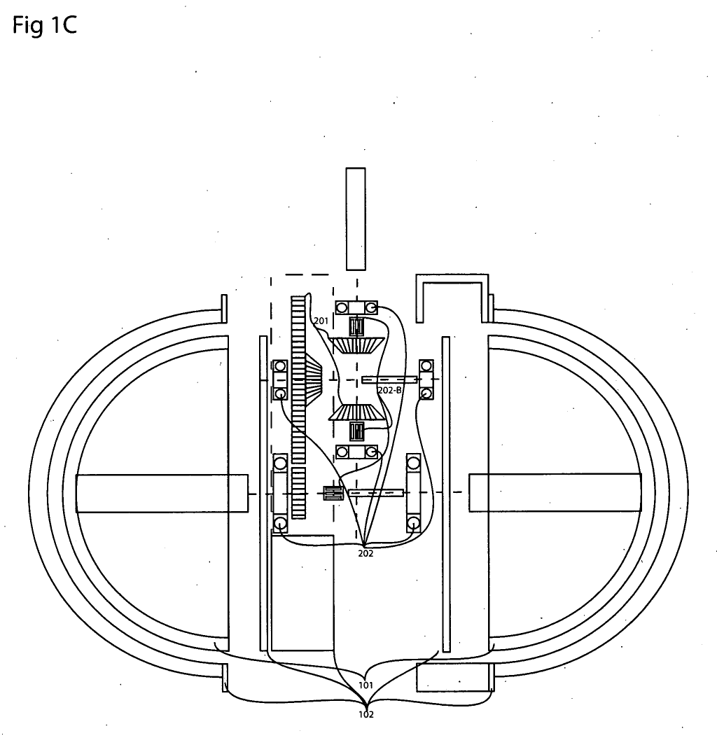

FIG. 1A is a detailed view of a Gyrokinetic Engine with drivetrain configuration within center of rotor and optional electricity generator

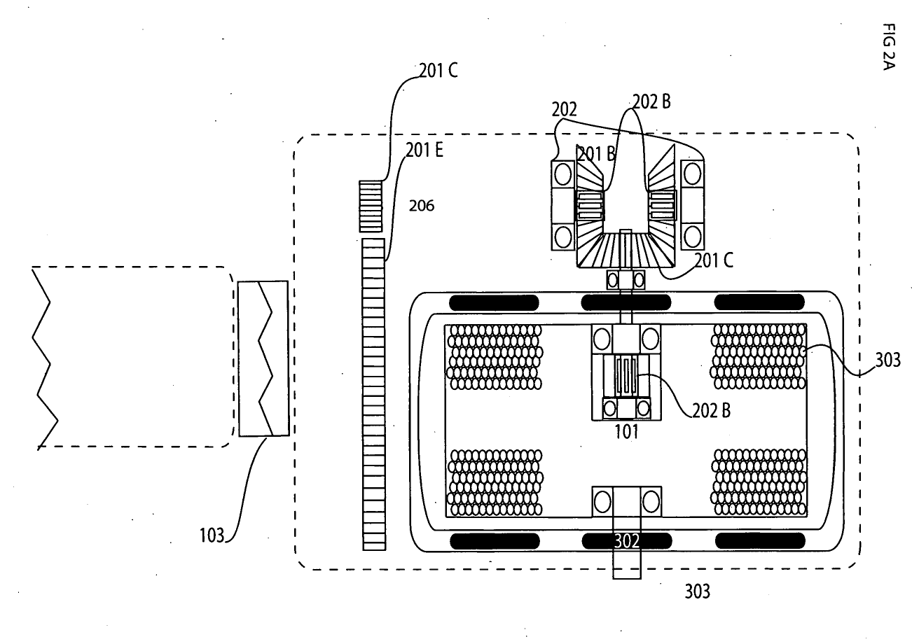

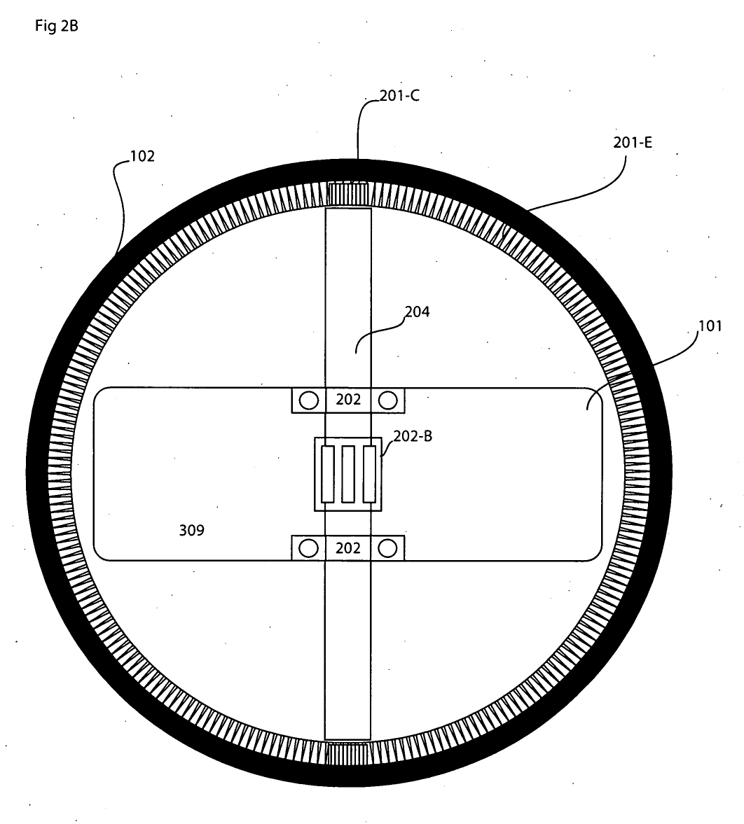

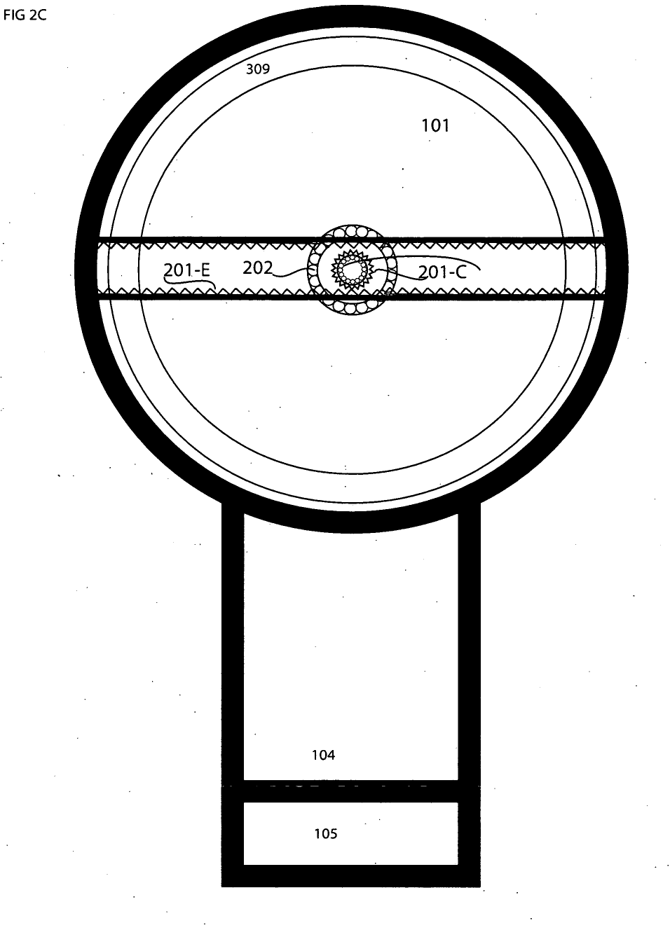

FIG. 1B is a detailed view of FIG. 2A assembled without optional equipment

FIG. 1C is a detailed view of FIG. 2A disassembled without optional equipment

FIG. 2A is a detailed view of an alternative drivetrain configuration for Gyrokinetic Engine in which the drivetrain components are outside of the rotor.

FIG. 2B is a detailed view of axial geared Gyrokinetic Engine drivetrain configuration in which the gear engagement is at the ends of axels with optional magnetic drive assist and electricity generator alternative embodiment

FIG. 2C is a side view of FIG. 2B

FIG. 2D is an illustration of a Gyrokinetic Engine with a variable ratio drivetrain

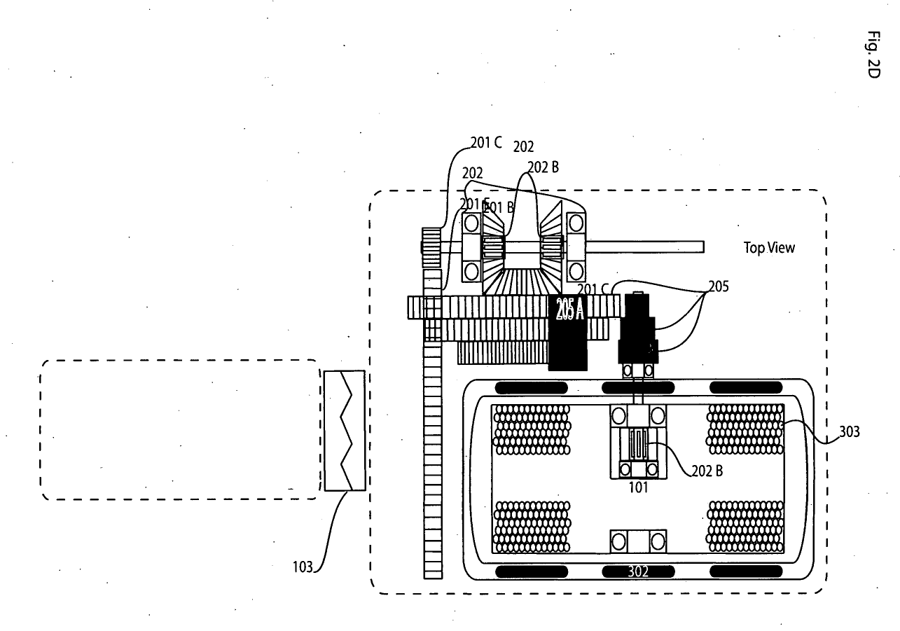

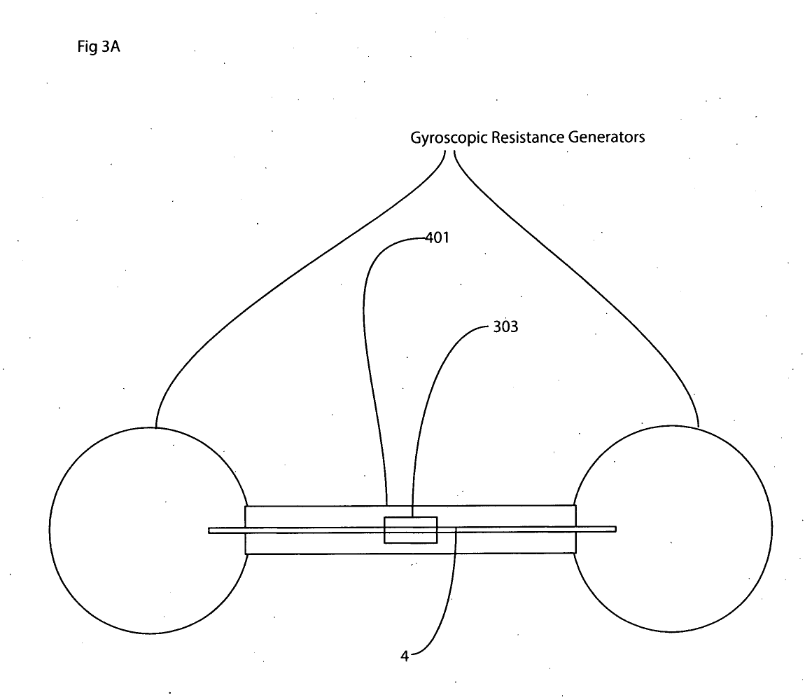

FIG. 3A is an illustration of an dual rotor, single grip alternative embodiment

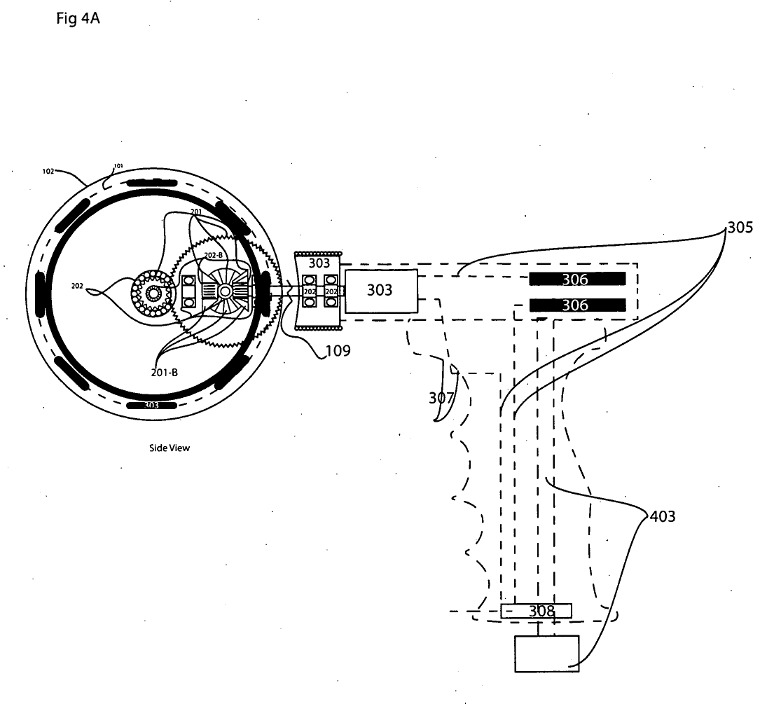

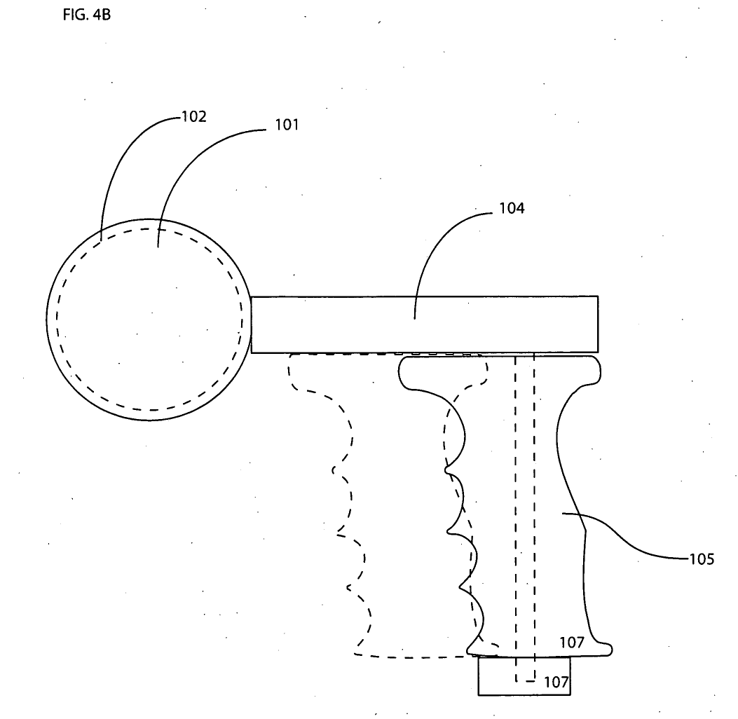

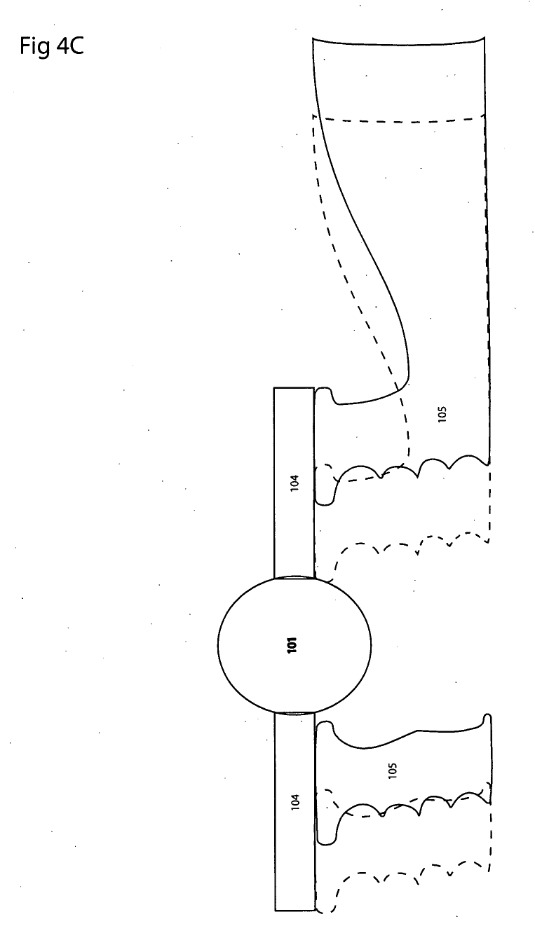

FIG. 4C is an illustration of a multiple grip, single rotor rifle alternative embodiment

FIG. 4A is an overall view of the present invention with pistol grip alternative embodiment and optional electricity generator and optional and optional motor drive.

FIG. 4B is an alternative embodiment of the pistol grip showing moveable grip position

### REFERENCE NUMERALS IN DRAWINGS

101 Rotating Mass
102 Frame
102-A Structural Component
103 Removable Coupling
104 Optional Attachment
105 Optional Grips or Handles
106 Optional Tightening knob
107 Optional Bolt
201 Gears
201-B Bevel Gear
201-C Pinion Gear
201-E Ring Gear
202 Bearings
202-B Clutch Bearing
204 Axle
205 Variable ratio pulleys
205-A Derailleur
301 Optional Electric motors
302 Optional Magnets
303 Optional Electromagnetic Coil
304 Programmable Microchip
304-C Memory
304-D Display screen
305 Optional Wiring and electronics
305-A Method of transmitting data
306 Optional Rechargeable Batteries
307 Optional Activation button/trigger/lever
308 Optional Laser Pointer
309 Material Providing mass
310 Rubber
311 Rotor Hub
314 Counterbalance Weight
401 Handle
403 Handle Clamping Bolt
500 Integrated Circuitry

### DETAILED SPECIFICATION OF THE PREFERRED EMBODIMENT

One exemplary embodiment of the present invention is illustrated in FIG. 1A which achieves a conversion of kinetic energy to angular momentum useful for providing resistance to movement through pivotally mounting a rotatably balanced mass 101 about a spin axis 204 to rotate freely then selectively engaged by a means of engaging rotation including clutching mechanism 202-B which is a one way clutch bearing to impart rotational torque unto rotating mass with increased rotational energy derived from the engagement of gears 201 turned through precession of the rotating mass caused by movement of the handles in such a way as to deflect the momentum causing the rotating mass to rotate faster and increasing the angular momentum and then disengaging to preserve momentum once the input rotation speed falls below that of rotating mass. This clutch engagement may occur within rotor as illustrated in FIGS. 1A, 1B, and 1C or at any place along the transmission of rotation between the engagement of precession to the rotating mass facilitated by any known means of engaging rotary motion including but not limited to one way clutch bearing as illustrated; centrifugal clutch; electromagnetic clutch; electromagnetic rotary engagement; pressure plate; hydraulic or gas means of transmitting rotational energy or actuating the engagement of engagement.

In this preferred embodiment illustrated in FIG. 1A the rotating mass 101 will be pivotally mounted by bearings 202 alongside a one way clutch bearing 202-B set to selectively engage the axle 204 to the rotating mass when it rotates faster than the rotating mass.

The axle 204 is turned by 201-C which is turned by ring gear 201-E which are in place to achieve an input ratio multiplying input rotation sufficiently to achieve angular momentum in quantities sufficient to sustain motion of device overcoming friction and providing resistance to the users movements which power the device. The ring gear is turned by engagement of the 201-B bevel gears which translate rotary motion through a 90 degree transition to align with the rotary engagement of the precession motion which occurs about an axis perpendicular to the rotation of said rotating mass. Two more 201-B Bevel gears are at the intersection with the bevel gear attached to the ring gear. These two additional bevel gears are mounted to one way clutch bearings set to engage rotation in opposite directions for the purpose of allowing input rotation to occur in either direction and be translated into a single direction of rotation by engaging the bevel gear on either side of the ring gears bevel gear which allows the user to move the exercise device in any direction of exercise. All rotary motion is supported by bearings 202 which are illustrated as ball bearings but could include any known means of reducing rotational friction including but not limited to oil bushings, air bearing, magnetic support and more. The Precession Engagement Shaft 206 does not rotate in drivetrain configurations illustrated in FIGS. 1A, 1B, C, and remains fixed with the handle while the rest of the frame rotates around shaft 206 with precession. In FIGS. 2A and 2D the precession of the rotating mass 101 moves shaft 206 around a ring gear that is in line with the path of precession rotation engaged by a pinion gear while the ring gear remains fixed with the handles. The frame which receives the pivotal mounting of rotating mass and gripping attachments will allow a means for precession rotation of the mass which has been constrained to it and so has a plurality of pivotal couplings to receive mounting of handles, attachments and other mounting configurations that would facilitating its use throughout its broad range of applications.

These pivotal couplings are placed at locations concentric to center of rotating mass at a distance great enough to allow precession within frame members at an axis perpendicular to the rotation of said mass where user interaction would best be engaged facilitating patterns of motion causing precession.

The rotary engagement of precession is illustrated as gears in these figures but could be accomplished by any known means of rotary engagement at the intersection between the stationary component such as the handle or mounting attachment and the precession of the rotating mass and transmitted by any known means of conveying rotation including belts, gears, hydraulically, pneumatically, and electromagnetically.

The rotary transmission motivated by a rotational engagement between grip or base of support and the precession of the rotating mass is constrained to travel only along the path where all components will align through frame 102 structures that allow said motion within and about them.

The bevel gears mounted to clutch bearings clutch are set to engage alternatively in opposite directions to translate bi-directional input rotation to unidirectional rotation output to rotating mass allowing unlimited increases of angular momentum through either direction of use. The engagement of these differential gears is optional and could be bypassed if the device was only to be used in one direction or the clutch bearings could be replaced with any other means of rotary engagement including electromagnetic engagement.

This device which is powered by user interaction through manipulation of the handles or mounting attachments moved circular or conical pattern of motion along any plane causing the rotating mass to precess while engaging the rotary transmission that will in turn cause the rotating mass to rotate at an increased rate set by the transmission ratio of input to output rotations. As the user causes an increase in the speed of the rotating mass and possesses increased angular momentum it will become increasingly difficult for the user to move the device and exercise with it creating progression in the exercise and achieving the intended goal of resisting motion.

A frame structure providing a with means for the rotating mass to rotate and precess about and axis perpendicular to its rotation with a means to accommodate the fitting of bearings to constrain the components to travel along their predefined path of motion while being rigid enough to prevent any additional motion. The Removable Coupling 103 will be suitable for attachment grips or shapes simulating the gripping of firearms or sporting goods facilitated by any known means of engagement including quick release rotary couplings of gas or fluid, bearing mounted protrusion with mating receptacle having locking clamp, screw in attachment of pivotally mounted structure, magnetic adhesion of pivotal components.

Attachments such as grips do not rotate as their held in the hand or otherwise restrained while precession turns the rotating mass element rotates.

### ALTERNATIVE EMBODIMENTS

In FIGS. 2B and 2C the axle of the rotating mass extends to engage a ring gear oriented along the path perpendicular to the precession rotation which is also perpendicular to the axis of rotation of the rotating mass. This axle has gear teeth at the ends to achieve positive engagement to ring gear as the rotating mass precesses these gears mesh causing rotating mass to rotate at an increased rate relative to the speed of precessions induced by user interaction with the device. The axle engagement is illustrated as toothed gear but could also include any known means of engagement. FIG. 2D illustrates a variable ratio transmission to allow the increase or decrease of the multiplication of precession rotations relative to rotations of rotating mass facilitated by a number of ring and pinion gears with varying tooth counts selected by any known means illustrated as a Derailleur 205-A and the rest of the device functions in the same manner as illustrated in FIG. 2A.

Alternative embodiments illustrated in FIGS. 1A, 2A, 2D, 4A show the rotating mass with optional magnets 302 positioned adjacent to electromagnetic coils 303 providing means to impart rotational energy through to starting, maintaining speed or increase rotational speed of rotating mass through activation of electromagnetic coils and alternatively when not electrically activated the electromagnetic coils will generate electricity while the rotating mass is motivated by user interaction or continuing through its own momentum.

An alternative embodiment illustrated in FIG. 4A illustrates how batteries 306 would be included to store electricity generated by the electromagnetic coil during use with a trigger 307 to return stored energy to electromagnetic coil to turn the rotating mass and a laser pointer 308 to aid in use of the device during marksmanship exercises and 403 clamp to facilitate movement of the grip along the length of the frame.

An alternative embodiment illustrated in FIGS. 2B and 2C demonstrates how the precession of the rotating mass could be engaged by gears placed at the end of an axle riding around a ring gear.

An alternative embodiment illustrated in FIG. 3A shows how the present invention could be located at either ends of a single handle 401.

An alternative embodiment illustrated FIG. 4A includes programmable microchip controller 304 which will control activation of electromagnetic coils 303 to regulate rotation of mass 101, regulate electricity generated by coils, and through various sensors 304 B measure speed of rotor, rotations of precession, and motions of user collected and stored in memory 304 C for display by screen 304 D or transmitted for use externally or to create game play control and interactive capabilities by a method of transmitting data 304.

Alternative embodiments could be driven any means of transmission of rotational energy including but not limited to shaft, belt, chain, fluid or gas conveyance of energy and any other known means to convey rotational energy.

Alternative embodiments could include a means of balancing about center of rotation including but not limited to harmonic balancing element, vibration absorbing hub, pillow mounting pivotal mounts, computer balanced mass, and any other known means to evenly distribute mass of a rotating object about its pivot.

Alternative embodiments could include any known means of transmitting single direction of rotation with ambiguous input rotation including differential gearing, hydraulic coupling controlled through valves controlling flow; electromagnetic conveyance, air pressure, and any other known means of accepting ambiguous rotation and outputting single direction of rotation.

Alternative embodiments could include any known means of pivotal attachment removable and means of receiving pivotal mounting including but not limited to removable rotatable pressurized gas and fluid couplings, bearing mounted electromagnetic coupling, quick release cam or clamping attachment of rotatably mounted mating parts, universal joint, flex shaft, and any other known means of rotatably coupling parts in a manner that allows intended motion while adhering to intended constraint.

Alternative embodiments could include any known means to accelerate said mass by electromagnetism that also generates electricity including all known means to create rotation through electromagnetism, electric motors or electrification of coils which also include a means for generating electricity through all known methods and techniques such as manually rotating an electric motor without power applied.

Alternative embodiments could include any known means of constantly variable speed-to-power ratio is achieved by including several sizes of gears arranged in a sequential manner to create desired ratios with a derailleur mechanism to move method of conveyance such as belt to chain onto the desired engagements

Alternative embodiments could include any known means of rotational engagement producing singular direction of rotation output from ambidextrous input rotation and Means to output a single direction of rotation accomplished by differentially opposed gears set on same input axle at either side of single output pinion engaged alternatively by one way clutch bearings set to engage in opposite directions. Thus each gear engages a different direction of rotation but on opposite sides of the pinion output single direction of rotation.

A method to resemble firearms or sporting equipment facilitated by modeling gripping attachment to resemble pistol, rifle, racquet, club, bat, hand tools and other instruments used by the hands.

A method of transmitting data would be accomplished by WiFi module, Bluetooth, and any other known means of wirelessly transmitting data connecting to source of data and power source

A method of sensing include gyroscope sensors, accelerometer, compass, optical sensors, hall effect sensor, temperature sensor, pressure sensing element, heart rate sensor and any other known sensors able to generate data to be transmitted connected to the transmitting sending constant stream of data to be received externally

A method of accelerating rotational speed would be accomplished by activation of electromagnetic coils coupled to magnetic elements within rotating body alternately oriented to produce rotation when coils are properly electronically activated in sequence.

### OPERATION—PREFERRED EMBODIMENT

Based on the particular intent and interest of the user, the grips 105 on the present invention are adjusted along its sliding rails 104 along its rotor housing attachment 103. The user is instructed to grasp the grips 105 as intended and place other hand on the housing and twist the device in a manner consistent with normal precession thus manually activating rotation of the rotor 101 to spin at a rate adequate to achieve precession and after precession is accomplished user would remove second hand and move the device in conical, circular or spinning motion based on the particular intent and interest of the user or attachment thus pushing the device against resistance produced and through its cycles of precession thus engaging the gears turning the rotor to spin said rotor at progressively increasing velocity.

As the device is pushed through its cycles of precession the gears mounted in the rotating housing 102 turn against fixed gear 201 mounted to handle or grip 105. As the housing revolves around its stationary mounting through cycles of precession the stationary gears 201 connected fixedly to the grip are engaged by the gear 201 as it revolves around it transmitting rotation to the next larger gear 201 in the drivetrain which then turns the next smaller gear 201 in the drivetrain attached to the axle of rotor 101 spinning the rotor 101 at a rate adequate to increase angular momentum at a rate proportionate to the cycles of precession and produce an amount of force in relative to energy input by the user.

A means to precess in either direction while conveying a single direction of torque rotation is accomplished by engagement of one of two gears are alternatively engaged at either side of rotating gear mounted on one way clutch bearings set in opposite directions for each gear set so that rotation is engage one at a time when device is moving in either direction as a single bearing and single stationary gear will engage the rotating gear and turn the drivetrain while the other will spin freely. Then once the device is moving in the other direction the first gear will disengage and the second set on the opposite side of the revolving gear will engage producing a consistent direction of rotation for the rotating gear regardless of direction of input motion. With stationary gears set on either side of rotating gear only one gear will engage in each direction of rotation at opposing sides of the rotating gear to produce a consistent direction of rotor spin with either direction of input.

The rotating gear will turn the next gear which is larger to increase the number of rotations of the smaller gear in the drivetrain which is turned by it achieving a spin ratio adequate for each cycle of precession. The final drive gear attached to the rotor axle engages through another one way clutch bearing intended to release the rotor to spin freely once user input has ceased and thus maintaining the angular momentum generated by user for later use.

Should the user need assistance maintaining the spin of the rotor 101, the user will be instructed to press the button 304 activating the electromagnetic motor 301 thereby inducing coupling of magnets embedded into the rotor 203 accelerating the spin rate of rotor 101, aiding the user in the exercise performance and providing support for normal operation. This significantly reducing or even negating the possibility that the user will become frustrated and give up doing the exercise as users normally would with prior art.

When the electromagnets are not activated and the user is powering the device through normal operation via precession engagement of gear system the electromagnets 303 will generate electricity as the solid state magnets 302 rotate with rotor 101 which will be stored in batteries 306 for later use to power the device or to be used externally.

### CONCLUSION, RAMIFICATIONS AND SCOPE

There is a multitude of neuromuscular, cardiorespiratory, circulatory, and performance benefits to be gained through regular performance of angular momentum resistance exercises which have never been realized with prior art due to its short lifespan, inconsistent performance and high failure rate of friction drive precession engines comprising all prior art.

The present invention embodies significant improvements over prior art producing a high performance kinetic engine capable of withstanding rigorous high intensity use by powerful athletes in rugged unpredictable environments and then seamlessly transitioning into the hands of a child or elderly person for a gentle cardiorespiratory leisure activity or game.

The preferred embodiment of the present invention has achieved levels of performance never before seen with any friction drive kinetic engines by nearly eliminating the constant drag of friction while enhancing the drive mechanism this device has achieved greater max power output, lower minimum power output, longer lifespan, reduced maintenance, and greater resistance to shock or contamination making it suitable for use throughout all environmental conditions.

There are many physical, neurological, and neuromuscular benefits that can be derived by mankind from performing gyroscopic exercises but, due to the excessive effort, steep learning curve, and rapid failure brought about by the shortcomings of friction driven rotors in prior art, this gift of greater health and capability has not been reached by a broader section of the population.

The present invention changes seeks to address and resolve this situation with significant improvements to prior art. By the reinventing the drive system gyroscopic exercise design is elevated to a level of functionality capable of meeting the needs of an ever evolving society, and combining this with a system able to assist challenged users, it's been made possible for almost anyone to perform these exercises on a daily basis without much training or assistance. With these advancements in exercise technology I hope to contribute in a small but significant way to the evolution of exercise design and the resulting advancement of capabilities and health.

I claim:

1) A method for producing resistance to motive forces through conversion of expressed kinetic energy, comprising
a) providing a device comprised of a gripping attachment; a frame structure; a method of receiving pivotal coupling of said gripping attachments to frame; a pivotal mounting to receive a rotating mass of predetermined specifications; area to accommodate said mass; a rotating mass possessing enough angular momentum to forcefully precess; a method of rotary engagement of precession; a method of transmission of said rotary engagement to said precession to rotation of said rotating mass;
b) providing an input of kinetic energy expressed through movement of gripping attachments with force greater than resistance to said movement whereby said method of producing resistance to motive forces through conversion of expressed kinetic energy begins by receiving said kinetic energy through said gripping attachments pivotally attached to said frame moved through a pattern of motion that would cause continual precession of said rotating mass pivotally coupled to frame and thus turning said rotary engagement by said precession which is transmitted through said transmission to further accelerate said rotating mass thus producing increased resistance to further motive forces.

2) the method for producing resistance to motive forces through conversion of expressed kinetic energy of claim 1 wherein user moves said device through said pattern in an alternate direction reversing rotation of said rotary engagement and said transmission which further includes a method to produce singular direction of output rotation to said rotating mass with ambidextrous rotation input

3) the method for producing resistance to motive forces through conversion of expressed kinetic energy of claim 1 further including a method to selectively engage accelerating rotational input allowing free rotation of rotating mass in the absence of accelerating force

4) the method for producing resistance to motive forces through conversion of expressed kinetic energy of claim 1 further including electromagnetic coils, magnetic elements and a method of accelerating rotational speed of said rotating mass through timed sequential activation of electromagnetic coils propelling said corresponding magnetic elements within rotating mass and then alternatively generating electricity by the rotation of mass when coils are not electrically activated

5) the method for producing resistance to motive forces through conversion of expressed kinetic energy of claim 1 further including a method to self-balance rotating mass

6) the method for producing resistance to motive forces through conversion of expressed kinetic energy of claim 1 further including a method of adjusting mass of rotating body

7) the method for producing resistance to motive forces through conversion of expressed kinetic energy of claim 1 further including a method of sensing, displaying and transmitting data externally the motion; rotation; heart rate; and all other available data and biometrics

8) the method for producing resistance to motive forces through conversion of expressed kinetic energy of claim 1 wherein gripping attachments have a method to resemble firearms or sporting equipment

9) A Gyrokinetic Engine powered by kinetic energy, comprising: a pivotally mounted rotatably balanced mass with means to rotate about axis of rotation and precess about the perpendicular axis; a frame with means to accommodate rotating mass providing for a plurality of pivotal couplings to gripping attachments or bases; a rotational energy coupling element with means of engaging rotation between the frame and coupled grip; and a means of transmitting said rotational energy to rotation of said mass, wherein said handle is twisted against said frame initiating rotation of mass and then held during movement of device in a manner to perpetuation precession of said rotating mass thereby engaging said coupling elements and transmitting said precession rotation to rotation of said mass and overcoming the resistance produced and successfully completing activity.

10) the Gyrokinetic Engine of claim 9 further including a means of adjusting quantity of rotating mass

11) the Gyrokinetic Engine of claim 9 further including a means for self-balancing

12) the Gyrokinetic Engine of claim 9 wherein rotational energy transmitted to rotation of said mass will have means to selectively engage when the rotational speed of input is greater than that of the rotating mass

13) the Gyrokinetic Engine of claim 9 wherein the means of transmission further includes a means to accept input from either direction of rotation while outputting one singe direction of rotation for rotating said mass

14) the Gyrokinetic Engine motor of claim 9 wherein the rotating mass further includes a composition of magnetic elements oriented with alternating polarities about the perimeter adjacent to corresponding electromagnetic coils with means to imparting rotational energy unto said rotating mass by sequential electrification of said coils which then generate electricity from rotation of mass when not electrified

15) a gyroscopic exercise device of the type with a rotating mass as the source of resistance for exercise is improved by providing said rotating mass with a means to selectively engage accelerating precession rotation and transmit it to the rotation of said mass

16) the gyroscopic exercise device of claim 15 wherein said accelerating precession is engaged by a toothed means of rotary engagement

17) the gyroscopic exercise device of claim 15 wherein input of said accelerating precession is conveyed by means of a variable speed-to-power ratio

18) the gyroscopic exercise device of claim 15 wherein said engagement of accelerating precession further includes a means to precess in either direction while conveying a single direction of rotation to rotating mass

19) the gyroscopic exercise device of claim 15 further including means to accelerate said mass by electromagnetism that would alternatively generate electricity when not electrified to accelerate mass

20) the gyroscopic exercise device of claim 15 wherein said rotating mass further includes a means of adjusting amount of rotating mass

21) the gyroscopic exercise device of claim 15 further including a means for self-balancing
